(19) United States
(12) Patent Application Publication
(10) Pub. No.: US 2004/0156874 A1
(43) Pub. Date: Aug. 12, 2004

(54) Urea- a topical anti-inflammatory

(75) Inventors: Bradley P. Glassman, Fairfield, NJ (US); Dileep Bhagwat, Bronxville, NY (US); Daniel Glassman, Fairfield, NJ (US)

Correspondence Address: COVINGTON & BURLING, ATTN: PATENT DOCKETING, 1201 PENNSYLVANIA AVENUE, N.W., WASHINGTON, DC, 20004-2401, US

(73) Assignee: Bradley Pharmaceuticals, Inc.

(21) Appl. No.: 10/365,624

(22) Filed: Feb. 11, 2003

(51) Int. Cl. A61K 31/17; A61K 7/00

(52) U.S. Cl. 424/401; 514/588

(57) ABSTRACT

The method of treating inflammatory skin conditions with compositions containing urea as the sole active ingredient is described. Although the mechanism by which urea acts as an anti-inflammatory agent is unclear, this very beneficial property has many useful applications in treating inflammatory dermatological conditions.

### FIELD OF THE INVENTION

The present invention relates to the discovery that urea, a naturally occurring ubiquitous chemical in man has anti-inflammatory properties when applied topically to inflamed skin. Although the mechanism by which urea acts as an anti-inflammatory agent is unclear, this very beneficial property has many useful applications in treating dermatological conditions.

### BACKGROUND OF THE INVENTION

Urea has been long recognized as a cosmetic ingredient in formulations acting as a humectant and moisturizer. High concentrations of urea, such as 40%, are also known to have mild, antibacterial effect. At these strengths the antibacterial effects are said to be similar to those of antibiotics, with the further advantage that all the common organisms are susceptible and the possibility of resistant strains need not be seriously considered. There have been reports of keratolytic activity attributed to urea with the ability at high concentrations to solubilize and denature protein. Dermatological compositions containing from 21 to 40 wt-% urea for treating dry scaly skin have been described in U.S. Pat. No. 5,919,470.

Concentrated solutions of urea can change the conformation of protein molecules. A striking effect is upon the water-binding capacity of the horny layer of the skin: pieces of normal horny layer, or scales from ichthyotic or psoriatic skin that have been soaked in 30% urea solution take up much more water. This is important because in maintaining the flexibility of the horny layer and the softness of the skin, the water content of the horny layer matters much more than its oil content.

Systemic drug therapy is associated with potentially harmful side effects. For example, oral anti-inflammatory drugs, for example corticosteroids, are distributed throughout the entire body. Systemic side effects such as elevated liver enzymes, gastrointestinal disorders and skin rashes are not uncommon and may require expensive intervention and laboratory tests. Accordingly, topical formulations for treating dermatological conditions of the skin in humans are increasingly recommended. Thus, topical compositions are generally preferred for dermatological applications. See, for example, “Practical Advice Offered On Rosacea”, Dermatology News, (April, 1985).

### SUMMARY OF THE INVENTION

Topical urea, formulated in a pharmaceutically acceptable base has surprisingly shown improvement in dermal inflammation. Inflammation is a local response to cellular injury that is marked by capillary dilatation, leukocytic infiltration, redness, heat, and pain and that serves as a mechanism initiating the elimination of noxious agents and of damaged tissue. The mechanism by which urea acts as an anti-inflammatory agent is unclear.

The present invention relates to the discovery that urea, a naturally occurring ubiquitous chemical in man, has anti-inflammatory properties when applied topically to inflamed skin. Urea can be applied to the affected dermal site formulated as, for example, a cream, lotion, solution, gel, lacquer, ointment, foam, or any vehicle capable of applying urea directly to the affected site.

Accordingly, the present invention provides a method for treating inflammation on the skin of humans and animals in need thereof by topically administering a safe and effective anti-inflammatory amount of urea in a pharmaceutically acceptable carrier.

### DETAILED DESCRIPTION OF THE INVENTION

Inflammation is a local response to cellular injury that is marked by capillary dilatation, leukocytic infiltration, redness, heat, and pain and that serves as a mechanism initiating the elimination of noxious agents and of damaged tissue. A human or animal must defend itself against multitude of different pathogens including viruses, bacteria, fungi, and protozoan and metazoan parasites as well as tumors and a number of various harmful agents which are capable to derange its homeostasis. For this, a plenty of effector mechanisms capable of defending the body against such antigens and agents have developed and these can be mediated by soluble molecules or by cells. If infection occurs as a consequence of the tissue damage, the innate and, later, the adaptive immune systems are triggered to destroy the infectious agent.

Inflammation is a complex stereotypical reaction of the body expressing the response to damage of its cells and vascularized tissues. In avascular tissues, e.g. in normal cornea, the true inflammation does not occur.

The discovery of the detailed processes of inflammation has revealed a close relationship between inflammation and the immune response.

The five basic symptoms of inflammation—redness (rubor), swelling (tumor), heat (calor), pain (dolor) and deranged function (functio laesa) have been known since the ancient Greek and Roman era. These signs are due to extravasation of plasma and infiltration of leukocytes into the site of inflammation. Early investigators considered inflammation a primary host defense system. From this point of view inflammation is the key reaction of the innate immune response but in fact, inflammation is more than this, since it can lead to death, as in anaphylactic shock, or debilitating diseases, as in arthritis and gout.

According to different criteria, inflammatory responses can be divided into several categories. The criteria include:

1. time—hyperacute (peracute), acute, subacute, and chronic inflammation;

2. the main inflammatory manifestation—alteration, exudation, proliferation;

3. the degree of tissue damage—superficial, profound (bordered, not bordered);

4. characteristic picture—nonspecific, specific;

5. immunopathological mechanisms

allergic (reaginic) inflammation,

inflammation mediated by cytotoxic antibodies,

inflammation mediaded by immune complexes,

delayed-type hypersensitivity reactions.

Inflammation is the body's reaction to invasion by an infectious agent, antigen challenge or even just physical, chemical or traumatic damage.

The mechanism for triggering the response the body to injury is extremely sensitive. Responses are to tissue damage that might not normally be thought of as injury, for example when the skin is stroked quite firmly or if some pressure is applied to a tissue. In addition, the body has the capacity to respond to both minor injuries such as bruising, scratching, cuts, and abrasions, as well as to major injuries such as severe bums and amputation of limbs.

Depending on the severity of the tissue damage resulting from an injury, the integrity of the skin or internal surfaces may be breached and damage to the underlying connective tissue and muscle, as well as blood vessels can occur. In this situation infection can, and frequently does result because the normal barrier to the entry of harmful organisms has been broken. It is obviously most important that the body can respond to injury by healing and repairing the damaged tissue, as well as by eliminating the infectious agents that may have entered the wound and their toxins. It is also important that the appropriate response to the tissue damage and infection can be made: it is no use bringing all of the body's defenses into action to repair a minor scratch, just as one would not expect a single mechanism to be able to deal with the sudden loss of a limb or a major infection.

The inflammatory reaction is phylogenetically and ontogenetically the oldest defense mechanism. The cells of the immune system are widely distributed throughout the body, but if an infection or tissue damage occurs it is necessary to concentrate them and their products at the site of damage. Three major events occur during this response:

1. An increased blood supply to the tissue “in danger”. It is performed by vasodilation. The inflamed tissue looks like containing greater number of vessels.

2. Increased capillary permeability caused by retraction of the endothelial cells. This permit larger molecules than usual to escape from the capillaries, and thus allows the soluble mediators of immunity to reach the site of inflammation.

3. Leukocytes migrate out of the capillaries into the surrounding tissues. In the earliest stages of inflammation, neutrophils are particularly prevalent, but later monocytes and lymphocytes also migrate towards the site of infection.

For the possibility of surrounding tissue damage, inflammatory responses must be well ordered and controlled. The body must be able to act quickly in some situations, for example to reduce or stop the lost of blood, whereas tissue repair and reconstruction can begin a little later. Therefore, a wide variety of interconnected cellular and humoral (soluble) mechanisms are activated when tissue damage and infection occur. On the other hand if the injury is negligible, the body must have mechanisms which are able to stop the tissue damage when the injury agent was removed.

The development of inflammatory reactions is controlled by cytokines, by products of the plasma enzyme systems (complement, the coagulation clothing, kinin and fibrinolytic pathways), by lipid mediators (prostaglandins and leukotrienes) released from different cells, and by vasoactive mediators released from mast cells, basophils and platelets. These inflammatory mediators controlling different types of inflammatory reaction differ. Fast-acting mediators, such as vasoactive amines and the products of the kinin system, modulate the immediate response. Later, newly synthesized mediators such as leukotrienes are involved in the accumulation and activation of other cells. Once leukocytes have arrived at a site of inflammation, they release mediators which control the later accumulation and activation of other cells.

However, in inflammatory reactions initiated by the immune system, the ultimate control is exerted by the antigen itself, in the same way as it controls the immune response itself. For this reason, the cellular accumulation at the site of chronic infection, or in autoimmune reactions (where the antigen cannot ultimately be eradicated), is quite different from that at sites where the antigenic stimulus is rapidly cleared.

The present invention relates to the discovery that urea, a naturally occurring ubiquitous chemical in man, has anti-inflammatory properties when applied topically to inflamed skin. Urea can be applied to the affected dermal site formulated as, for example, a cream, lotion, solution, gel, lacquer, ointment, foam or any other vehicle capable of applying urea directly to the affected site.

Such method includes administering to the affected skin of a human or animal with inflammation a safe and effective amount of urea, for example, from about 10 to 60 wt-%, preferably about 30-50 wt-%, and particularly about 40 wt-% of urea. The terms “administering” or “administration”, as needed herein, refer to any method which, in sound medical practice, delivers the urea, e.g., 40% urea, in such a manner so as to be effective in the treatment of inflamed dermatological disorders of the skin. The phase “safe and effective amount”, as used herein, means an amount of urea sufficient enough to significantly and positively modify the condition to be treated but low enough to avoid serious side effects, within the scope of sound medical advice. The safe and effective amount of the urea will vary with the particular pharmaceutically acceptable carriers utilized, and the like factors within the knowledge and expertise of the attending physician.

The method of the present invention typically involves administering the urea in an amount to cover the affected area. The specific preferred quantity of the urea depends upon the characteristics of the dermatological disorder.

The dosing of the compositions of this invention may be for acute conditions or chronic conditions. However, it has been found that urea is particularly effective when the inflammation is due to microbial infection.

For the method of the present invention, the duration of administration of the urea will vary according to the specific extent of the dermatological condition being treated and if the treatment requirements are for acute or chronic therapy.

Another embodiment of the invention is a method for using urea therapeutically as an anti-inflammatory to treat dermatological disorders of the skin in humans and animals by topically administering a safe and effective amount of urea in a pharmaceutically acceptable carrier.

In addition to containing a therapeutically effective amount of urea the composition includes dermatologically acceptable excipients as described in U.S. Pat. No. 5,919,470, which patent is incorporated herein by reference. The excipients particularly include skin protectants which include a combination of semi-solid and liquid petroleum fractions. The semi-solid skin protectant is contained in about 5.5 to about 20 wt-% and includes petrolatum or a synthetic or semi-synthetic hydrocarbon of the same nature as petrolatum. Mixtures of such ingredients can also be used. The preferred semi-solid material is petrolatum, commercially available from a wide variety of sources.

The liquid portion skin protectant is a liquid petrolatum and contained in the composition in about 10 to about 20 wt-%. This material can include any synthetic or semi-synthetic oleaginous liquid fraction. A preferred embodiment is mineral oil, which is a liquid mixture of hydrocarbons obtained from petroleum.

Another preferred ingredient encompassed in the composition of the present invention is propylene glycol which may be contained up to about 5 wt-% in the composition, preferably in the range of from about 1 to about 5 wt-%.

Typical compositions containing urea employed in the present invention are for example:

| Ingredient | Approximate Wt-% |
|---|---|
| Urea | 40 |
| Petrolatum or a synthetic or semi-synthetic | 5.5-20 |
| hydrocarbon, or a semi-solid mixture thereof | |
| liquid petrolatum or synthetic or semi-synthetic | 10-20 |
| oleaginous liquid fraction, or a mixture thereof | |
| C16-18 aliphatic straight or branched chain fatty | 0.25-2 |
| alcohol or fatty acid, or a mixture thereof | |
| propylene glycol | 1-5 |
| glyceryl stearate | 1-3 |
| xanthan gum | 0.01-0.5 |
| water QS | 100.0 |
| Urea | 30 |
| Petrolatum or a synthetic or semi-synthetic | 5.5-20 |
| hydrocarbon, or a semi-solid mixture thereof | |
| liquid petrolatum or a synthetic or semi-synthetic | 10-20 |
| oleaginous liquid fraction, or a mixture thereof | |
| C₁₆₋₁₈ aliphatic straight or branched chain fatty | 0.25-2 |
| alcohol or fatty acid, or a mixture thereof | |
| propylene glycol | 1-5 |
| glyceryl stearate | 1-3 |
| xanthan gum | 0.01-0.5 |
| mixture of a carbomer and triethanolamine | 0.05-30 |
| Water QS | 100.0 |

### EXAMPLE

A typical formulation representing the particular and most preferred cream embodiment of the present invention is illustrated as follows:

| Ingredient | % W/W |
|---|---|
| Purified water | 36.149 |
| Urea USP | 40.000 |
| Carbopol 940 | 0.25 |
| Petrolatum | 5.94 |
| Mineral oil | 12.06 |
| Glyceryl stearate | 1.875 |
| Cetyl alcohol | 0.626 |
| Propylene glycol | 3.00 |
| Xanthan gum | 0.050 |
| Trolamine NF | 0.150 |
| TOTAL QS | 100.00 |

The above ingredients were mixed together to form a cream in accordance with conventional, commercially known methods.

We claim:

1. A method of treating topical inflammatory conditions of the skin comprising administering to the affected area of a patient in need thereof a composition consisting of an anti-inflammatory effective amount of urea and a balance of dermatologically acceptable excipients.

2. The method of claim 1, wherein the composition comprises from about 10 to about 60 wt-% urea.

3. The method of claim 1, wherein the composition comprises from about 30 to 50 wt-% urea.

4. The method of claim 1, wherein the composition comprises about 40 wt-% urea.

5. The method of claim 1, wherein the composition is selected from the group consisting of topical creams, ointments, solutions, lacquers, gels, foams and any other vehicle that can be applied directly to the affected area of the skin.

6. A method of improving the topical dermatological inflammation conditions of the skin comprising administering to the affected area of a patient in need thereof a composition comprising:
(a) about 30 to about 50 wt-% urea;
(b) about 5.5 to about 20 wt-% petrolatum or a synthetic or semi-synthetic hydrocarbon, or a semi-solid mixture thereof;
(c) about 10 to about 20 wt-% of a liquid petrolatum or a synthetic or semi-synthetic oleaginous liquid fraction, or a mixture thereof;
(d) about 0.25 to about 2 wt-% of a C₁₆₋₁₈ aliphatic straight or branched chain fatty alcohol or fatty acid, or a mixture thereof;
(e) about 1 to about 5 wt-% propylene glycol;
(f) about 1 to about 3 wt-% glyceryl stearate;
(g) about 0.01 to about 0.5 wt-% xanthan gum; and
(h) the balance being water.

7. The method of claim 6, wherein the composition further comprises about 0.05 to about 30 wt-% of a mixture of a carbomer and triethanolamine.

8. The method of claim 6, wherein the composition comprises about 40 wt-% urea.
